(19) United States
(12) Patent Application Publication
(10) Pub. No.: US 2012/0232262 A1
(43) Pub. Date: Sep. 13, 2012

(54) METHOD FOR PREPARING N-SUCCINIMIDYL N-BIOTINYL-6-AMINOCAPROATE

(75) Inventors: Pierre Potier, Paris (FR); Raphael Sole, Paris (FR); Patrick Trouilleux, Paris (FR)

(73) Assignee: SANOFI, Paris (FR)

(21) Appl. No.: 13/510,900

(22) PCT Filed: Nov. 19, 2010
(86) PCT No.: PCT/FR2010/052452

§ 371 Date: May 18, 2012

(30) Foreign Application Priority Data
Nov. 20, 2009 (EP) 0905573

(51) Int. Cl. C07D 235/00; C07H 1/00; C08B 37/00

(52) U.S. Cl. 536/55.1; 548/304.1

(57) ABSTRACT

The invention relates to a method for preparing N-succinimidyl N-biotinyl-6-aminocaproate, characterised in that said method includes a step of activating N-biotinyl-6-aminocaproic acid in the form of a mixed anhydride, followed by coupling said mixed anhydride with N-hydroxysuccinimide.

The present invention relates to a novel process for preparing the biotinylation reagent N-succinimidyl N-biotinyl-6-aminocaproate, and also to a process for preparing biotinylated polysaccharides using such a reagent.

N-Succinimidyl N-biotinyl-6-aminocaproate is a commercially available biotinylation reagent used in particular for grafting the biotin group onto DNA molecules or glycoproteins.

Given the labor and raw material costs, and in order to obtain N-succinimidyl N-biotinyl-6-aminocaproate on an industrial scale, it is necessary to envision a robust synthesis which is suitable for the production of the desired compound in large amounts and which makes it possible to obtain the latter in sufficiently pure form, i.e. without the formation of undesirable by-products. The inventors have now found a route of access to N-succinimidyl N-biotinyl-6-aminocaproate in two main stages starting from biotin, which meets the abovementioned requirements.

The process according to the invention comprises the stages represented below in scheme 1, in which R represents an alkyl or aryl group, and X and X′, which may be identical to or different than one another, represent halogen atoms.

In scheme 1, the starting, intermediate and final compounds are the following:
compound (I): N-succinimidyl N-biotinyl-6-aminocaproate,
compound (II): N-biotinyl-6-aminocaproic acid,
compound (II′): N-biotinyl-6-aminocaproate carboxylate,
compound (III): biotin,
compound (III′): cyanomethyl biotinate.

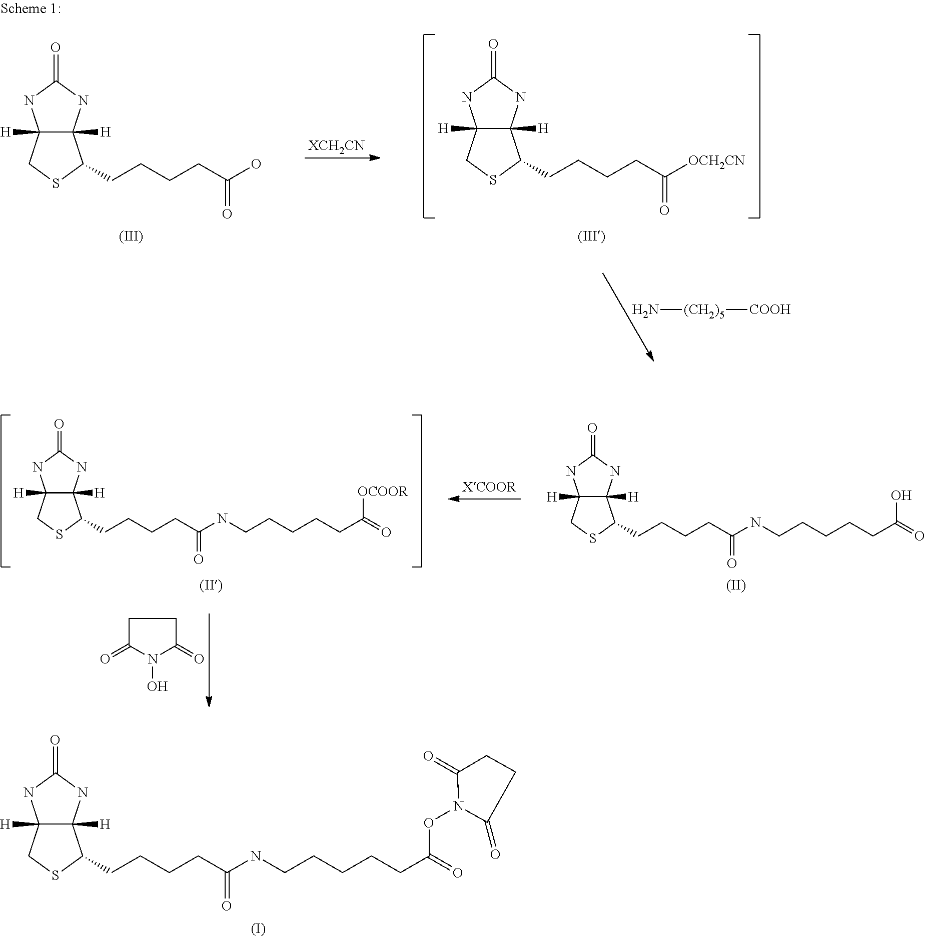

According to the present invention, and unless otherwise mentioned in the text:
the term “alkyl” is intended to mean: a linear or branched, saturated or unsaturated, hydrocarbon-based aliphatic group containing, for example, from 1 to 6 carbon atoms, advantageously from 1 to 4 carbon atoms,
the term “aryl” is intended to mean: a cyclic aromatic group, for example a phenyl group, and
the term “halogen” is intended to mean: a fluorine, chlorine, bromine or iodine atom.

The subject of the invention is thus a process for preparing the compound (I), characterized in that it comprises a stage of coupling the compound (II′) with N-hydroxysuccinimide. This coupling is advantageously carried out at a temperature below 0° C., for example at approximately −5° C.

According to the invention, the compound (II′) is obtained by treating the compound (II) with an alkyl or aryl haloformate (X′COOR, where R represents an alkyl or aryl group and X′ a halogen atom) in the presence of a weak base of tertiary amine type, in a polar and aprotic solvent, for example DMF (dimethylformamide), DMSO (dimethyl sulfoxide) or NMP (N-methylpyrrolidone). This reaction makes it possible to activate, in the form of a mixed anhydride, the acid function of the compound (II). It is advantageously carried out at a temperature below 0° C., for example at approximately −5° C.

Ethyl chloroformate (ClCOOEt) is advantageously used to obtain the compound (II′). The reaction is advantageously carried out in the presence of triethylamine (NEt₃) as weak base of tertiary amine type, and in a solvent such as DMF.

According to the invention, the compound (II) is obtained by means of a stage of coupling between the compound (III′) and aminocaproic acid (H₂N—(CH₂)₅—COOH). During this stage, a polar solvent, for example DMF, DMSO or NMP is advantageously used. The reaction is advantageously carried out under hot conditions, at approximately 100° C.

With regard to the compound (III′), it is obtained by treating the compound (III) with a haloacetonitrile (X—CH₂CN, where X represents a halogen atom), in the presence of a weak base of tertiary amine type, in a polar and aprotic solvent, for example DMF, DMSO or NMP. This reaction makes it possible to activate, in the form of an ester, the acid function of the compound (III). The reaction is advantageously carried out under hot conditions, at approximately 60° C.

Chloroacetonitrile (ClCH₂CN) is preferably used to obtain the compound (III′). The reaction is advantageously carried out in the presence of triethylamine as weak base of tertiary amine type, and in a solvent such as NMP.

According to the process of the present invention, the synthesis intermediates (II′) and (III′) are not isolated; thus, the reaction for preparing the compound (I) which is presented according to scheme 1 consists of a two-stage reaction starting from biotin.

Particularly advantageously, the process according to the invention makes it possible to readily gain access to the compound (II) in one stage starting from biotin and aminocaproic acid, with a high yield (greater than 80%) and resulting in a product of excellent purity.

Surprisingly, other routes of access to the compound (II) which could have been envisioned, inspired by coupling techniques resulting from peptide synthesis, have proved to be inefficient or unsuitable for industrial production.

In particular, the inventors have shown that the acid chloride route, which consists in converting the biotin (III) into the acid chloride (for example, D. E. Wolf et al., in J. Am. Chem. Soc., year 1951, 73, p. 4142-4144 and year 1952, 74, p. 2002-2003), and then in reacting it with aminocaproic acid in a basic medium, does not make it possible to obtain a satisfactory yield, owing to the insolubility of the biotin. Moreover, this route involves the use of a very large excess of thionyl chloride: the toxicity of this solvent causes industrial hygiene problems.

With regard to the dicyclohexylcarbodiimide (DCC) route, which consists in activating the biotin in situ before coupling it with aminocaproic acid (see, for example, C. Somlai et al., in Zeit. Naturforsch., 1993, 48, p. 511-516), it has proved to be inefficient owing to the insufficient reactivity between the activated biotin and aminocaproic acid. In addition, the use of DCC with aminocaproic acid generates DCU (dicyclohexylurea), a by-product which is very difficult to remove at the end of the reaction (DCU inseparable from the compound (II′)).

Finally, the mixed anhydride route (activation of the acid function of the biotin via the formation of a very reactive anhydride, which subsequently reacts with aminocaproic acid) makes it possible to obtain the desired compound (II), but has several drawbacks which make it unsuitable for the industrial scale: indeed, regardless of the conditions used, there is always some biotin remaining at the end of the reaction (at least 2%); a by-product forms, corresponding to the addition of 2 aminocaproic acids to the biotin, and the medium is rapidly converted into an extremely thick gel.

The activated ester route according to the present invention therefore overcomes the drawbacks of the other abovementioned routes for obtaining the compound (II).

Particularly advantageously, the process according to the invention makes it possible to gain access to the compound (I) in one stage starting from the intermediate of formula (II) and N-hydroxysuccinimide, according to the mixed anhydride synthesis route. This synthesis route makes it possible to obtain the compound (I) with a high purity and a high yield.

The subject of the invention is also a process for preparing biotinylated polysaccharides, for example idrabiotaparinux, characterized in that it comprises the following stages:
preparing the compound (I) according to the process defined above, then
coupling the compound (I) with a polysaccharide which has a function that is reactive toward the activated ester function of the compound (I).

Said reactive function of the polysaccharide is, for example, an amine function, in which case the coupling with the compound (I), which comprises an activated ester function, is carried out by means of a conventional amino/acid coupling reaction.

The coupling reaction between the compound (I) and the polysaccharide is advantageously carried out in an aqueous solution of sodium hydrogen carbonate.

The biotinylated polysaccharides, the preparation of which is described above, are for example such as those described in patent applications WO 02/24754 and WO 2006/030104. They may in particular be the biotinylated pentasaccharide known under the International Nonproprietary Name “idrabiotaparinux” and described in patent application WO 02/24754, or the biotinylated hexadecasaccharides described in examples 1 and 2 of patent application WO 2006/030104.

In order to prepare these biotinylated polysaccharides, the compound (I) is coupled, respectively, with the pentasaccharide 44 described in patent application WO 02/24754 or with the hexadecasaccharides 42 and 43 described in patent application WO 2006/030104:
pentasaccharide 44: methyl (2-amino-2-deoxy-3,4-di-O-methyl-6-O-sulfonato-α-D-glucopyranosyl)-(1→4)-(2,3-di-O-methyl-β-D-glucopyranosyluronic acid)-(1→4)-(2,3,6-tri-O-sulfonato-α-D-glucopyranosyl)-(1→4)-(2,3-di-O-methyl-α-L-idopyranosyluronic acid)-(1→4)-2,3,6-tri-O-sulfonato-α-D-glucopyranoside,
hexadecasaccharide 42: methyl (2,3,4,6-tetra-O-sulfonato-α-D-glucopyranosyl)-(1→4)-(2,3,6-tri-O-sulfonato-α-D-glucopyranosyl)-(1→4)-(2,3,6-tri-O-sulfonato-β-D-glucopyranosyl)-(1→4)-(2,3-di-O-methyl-6-O-sulfonato-α-D-glucopyranosyl)-(1→4)-(2,3,6-tri-O-methyl-β-D-glucopyranosyl)-(1→4)-[(2,3,6-tri-O-methyl-α-D-glucopyranosyl)-(1→4)-O-(2,3,6-tri-O-methyl-β-D-glucopyranosyl)-(1→4)]₃-(2-amino-2-deoxy-3-O-methyl-6-O-sulfonato-α-D-glucopyranosyl)-(1→4)-(2,3-di-O-methyl-β-D-glucopyranosyluronic acid)-(1→4)-(2,3,6-tri-O-sulfonato-α-D-glucopyranosyl)-(1→4)-(2,3-di-O-methyl-α-L-idopyranosyluronic acid)-(1→4)-3-O-methyl-2,6-di-O-sulfonato-α-D-glucopyranoside;
hexadecasaccharide 43: methyl (2,3,4,6-tetra-O-sulfonato-α-D-glucopyranosyl)-(1→4)-(2,3,6-tri-O-sulfonato-α-D-glucopyranosyl)-(1→4)-(2,3,6-tri-O-sulfonato-β-D-glucopyranosyl)-(1→4)-(2,3-di-O-methyl-6-O-sulfonato-α-D-glucopyranosyl)-(1→4)-(2,3,6-tri-O-methyl-β-D-glucopyranosyl)-(1→4)-[(2,3,6-tri-O-methyl-α-D-glucopyranosyl)-(1→4)-O-(2,3,6-tri-O-methyl-β-D-glucopyranosyl)-(1→4)]₃-(2-amino-2-deoxy-3-O-methyl-6-O-sulfonato-α-D-glucopyranosyl)-(1→4)-(2,3-di-O-methyl-β-D-glucopyranosyluronic acid)-(1→4)-(2,3,6-tri-O-sulfonato-α-D-glucopyranosyl)-(1→4)-(2,3-di-O-methyl-α-L-idopyranosyluronic acid)-(1→4)-2,3,6-tri-O-sulfonato-α-D-glucopyranoside.

The invention is illustrated by means of the examples which follow, which detail a process for preparing the compound (I) in accordance with the invention. In these examples, the following abbreviations are used: Ac: acetyl; LC: liquid chromatography; DMF: dimethylformamide; min: minutes; MTBE: methyl tert-butyl ether; NMP: N-methylpyrrolidone; eq: molar equivalents; Et: ethyl; T: temperature; THF: tetrahydrofuran; tR: retention time; OU: operating units; V: volume.

### EXAMPLE 1

### Preparation of the Compound (I)

The reactions are monitored by LC with the following conditions: Symmetry C18 150×4.6 mm 5μ column (Waters); eluent A: 0.01 M KH₂PO₄ buffer adjusted to pH=3; eluent B: acetonitrile; flow rate 1 ml/min; gradient: t=0 min A/B 85/15, t=9 min A/B 65/35, t=10 min A/B 85/15, t=15 min A/B 85/15. This method makes it possible to visualize the biotin (compound (III), tR=4.5 min), the intermediate activated ester (III′) (tR=8.4 min), the N-biotinyl-6-aminocaproic acid (compound (II), tR=5.5 to 5.6 min), the intermediate mixed anhydride (II′) (tR=11.2 min) and the N-succinimidyl N-biotinyl-6-aminocaproate (compound (I), tR=7.9 to 8.2 min).

### 1.1: Preparation of the Compound (II)

7.5 kg of biotin (III), triethylamine (15 l, 2 V, 3.5 eq), NMP (15 l, 2 V) and, finally, chloroacetonitrile (3.5 kg, 0.47 OU, 1.5 eq) are charged to a reactor. The medium is heated to 60° C. After this temperature has been maintained for 2 h, an LC analysis shows that all the biotin has been converted into compound (III′) (<2%). The medium is cooled to 50° C. and then transferred into another reactor, containing aminocaproic acid (9.05 kg, 1.206 OU, 2.2 eq). Rinsing is carried out with NMP (0.1 V). The medium is heated to 100° C. and maintained at this temperature for 2 h. An LC analysis shows that less than 2% of activated biotin (III′) remains. The medium is cooled to 60° C. Acetonitrile (60 l, 8 V) preheated to 55° C. is run in. The mixture is stirred for 30 minutes at 60° C., and then cooled to 20° C. Stirring is carried out for 1 h. The suspension is filtered, then rinsing is carried out with 3 times acetonitrile (5 V) and then with THF (5 V). Drying is carried out under vacuum at a maximum of 60° C. until there is no change in weight. 12.0 kg of the compound (II) are thus obtained, with a yield of 109% and an organic purity, measured by LC, of 98.6%.

10.0 kg of the compound (II) are recharged to a reactor. Hydrochloric acid (90 l, 9 V of water+10 l, 1 V of 36% HCl) is then added. The suspension is stirred at 20° C. for 30 min. The suspension is filtered and rinsing is carried out 3 times with water (4 V, 40 l), then twice with THF (3.5 V). Drying is carried out under vacuum at a maximum of 45° C. until there is no change in weight. 6.1 kg of the compound (II) are thus obtained, with a yield of 66%.

### 1.2: Preparation of the Compound (I)

In a reactor, 3 kg of the compound (II) are suspended in DMF (25 l, 8.3 V) and the temperature is brought to −5° C. Triethylamine (1.02 kg, 0.34 OU, 1.2 eq) is then added. After stirring for 15 minutes, ethyl chloroformate (1.1 kg, 0.365 OU, 1.2 eq) is added gently (over the course of at least 1 h). Rinsing is carried out with DMF (0.9 l, 0.3 V). The medium is stirred at −5° C. for at least 2 h. The suspension becomes finer and yellow. An LC analysis shows that all the compound (II) (<3%) has reacted.

N-Hydroxysuccinimide (1.04 kg, 0.386 OU, 1.2 eq) in solution in DMF (3 l, 1 V) is then introduced in 1 step (over the course of at least 20 min). Rinsing is carried out with DMF (1.5 l, 0.5 V). The medium is stirred for 1 h 30 at −5° C. An LC analysis shows that the presence of residual compound (II) is less than 3%. The temperature is brought to 22° C., the suspension is taken up in DCM (12 V, 36 l) and the resulting organic phase is washed with water (15 l, 5 V). The organic phase is drawn off and the aqueous phase is extracted twice with DCM (30 l, 3 V). The organic phases are mixed and are washed with water (1.5 l, 0.5 V). The organic phase is concentrated to 6 V, i.e. 181. Heating is carried out at 40° C. and MTBE (6.25 V, 19 l) is added over the course of a minimum of 1 h. The mixture is maintained at 40° C. for 1 h, and then MTBE (8.75 V, 26 l) is added over the course of a minimum of 2 h. The mixture is maintained at 40° C. for at least 30 minutes, and then cooled to 20° C. over the course of a minimum of 2 h, and maintained at this temperature for 30 minutes. The suspension is filtered by suction and the cake is washed with acetone (5 V, 15 l) and then twice more with acetone (2 V, 6 l). The resulting product is filtered by suction and dried in an oven under vacuum at a maximum of 40° C. until there is no change in weight.

3 kg of the compound (I) are thus obtained in the form of a cream powder, with a yield of 80% and an organic purity, measured by LC, of 96.0%. Except for the compound (II), the presence of which is not problematic for a subsequent coupling reaction with a polysaccharide since it will be inert during this coupling, the purity of the compound (I) is 98%.

### EXAMPLE 2

### Preparation of a biotinylated polysaccharide, idrabiotaparinux

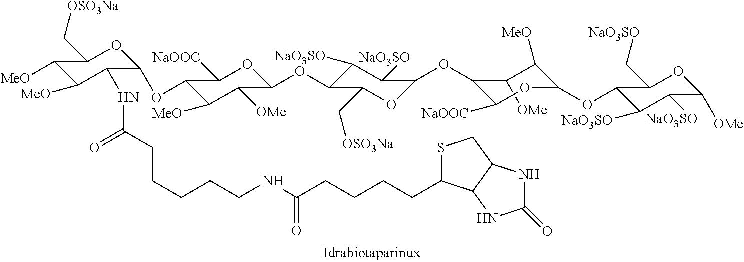

A solution of 1.22 kg of the crude pentasaccharide 44 (containing salts), as described in patent application WO 02/24754, is prepared in 8.51 of water (7 V). 0.5 kg (1.6 eq) of the compound (I), 0.12 kg (2.0 eq) of NaHCO₃ and 0.37 kg of NaCl are added thereto. The solution is in the form of a white suspension. 3.7 l of acetone are added thereto and the reaction medium is stirred at approximately 25° C. for at least 22 h. This suspension is then slowly run into a mixture of ethanol (120 l) and MTBE (60 l) cooled beforehand to approximately 4° C., which makes it possible to precipitate the biotinylated pentasaccharide. The resulting suspension is then filtered and rinsed successively with absolute ethanol and acetone. The precipitate is oven-dried under a vacuum until there is no change in weight. 1.60 kg of crude idrabiotaparinux (containing salts) are thus obtained in the form of a cream powder, with an organic purity of 99%, and with a yield of 109% with respect to the pentasaccharide 44 and a chemical yield of 70% over the last 3 stages.

1. A process for preparing N-succinimidyl N-biotinyl-6-amino caproate corresponding to formula (I) and comprising an activated ester function: 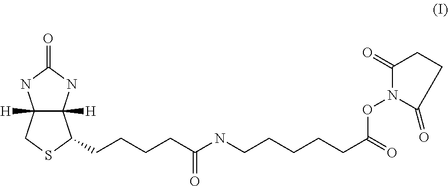
said process comprising a stage of activating, in the form of a mixed anhydride, the acid function of the compound (II): 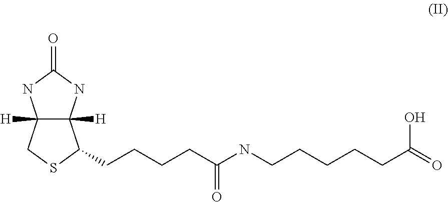
so as to obtain the compound (II′), in which R represents an alkyl or aryl group: 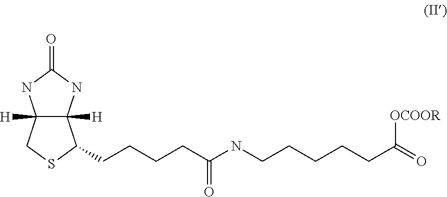
followed by coupling of the compound (II′) with N-hydroxysuccinimide.

2. The process as claimed in claim 1, wherein the activation of the acid function of the compound (II) is carried out by treating said compound with an alkyl or aryl haloformate of formula X′COOR, where R represents an alkyl or aryl group and X′ represents a halogen atom, in the presence of a weak base of tertiary amine type, in a polar and aprotic solvent.

3. The process as claimed in claim 2, wherein said haloformate of formula X′COOR is ethyl chloroformate.

4. The process as claimed in claim 2, wherein said weak base of tertiary amine type is triethylamine.

5. The process as claimed in claim 2, wherein said polar and aprotic solvent is selected from dimethylformamide, dimethyl sulfoxide or N-methylpyrrolidone.

6. The process as claimed in claim 1, wherein the stage of activating the acid function of the compound (II) is carried out at a temperature below 0° C.

7. The process as claimed in claim 1, wherein the compound (II) is obtained by means of a step of activating, in the form of an ester, the acid function of the compound (III): 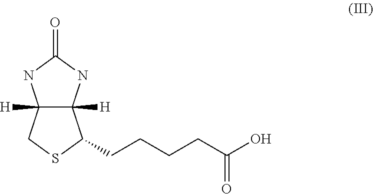
so as to obtain the compound (III′): 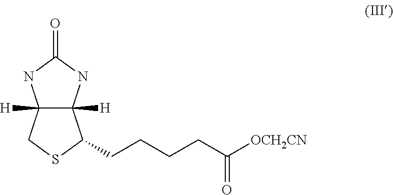
followed by coupling of the compound (III′) with aminocaproic acid.

8. The process as claimed in claim 7, wherein the activation of the acid function of the compound (III) is carried out using a haloacetonitrile, in the presence of a weak base of tertiary amine type, in a polar and aprotic solvent.

9. The process as claimed in claim 8, wherein said haloacetonitrile is chloroacetonitrile.

10. The process as claimed in claim 8, wherein said weak base of tertiary amine type is triethylamine.

11. The process as claimed in claim 8, wherein said polar and aprotic solvent is selected from dimethylformamide, dimethyl sulfoxide or N-methylpyrrolidone.

12. The process as claimed in claim 7, wherein the reaction for activation of the acid function of the compound (III) is carried out at approximately 60° C.

13. The process as claimed in claim 7, wherein the coupling reaction between the compound (III′) and aminocaproic acid is carried out at approximately 100° C.

14. A process for preparing biotinylated polysaccharides, comprising the following stages:
preparing the compound (I) according to the process defined in claim 1, then
coupling the compound (I) with a polysaccharide which has a function that is reactive toward the activated ester function of the compound (I).

15. The process as claimed in claim 14, wherein said reactive function of the polysaccharide is an amine function.

16. The process as claimed in claim 14, wherein it consists of the preparation of idrabiotaparinux and comprises the following stages:
preparing the compound (I) according to the process defined in claim 1, then
coupling the compound (I) with the sodium salt of the pentasaccharide methyl (2-amino-2-deoxy-3,4-di-O-methyl-6-O-sulfonato-α-D-glucopyranosyl)-(1→4)-(2,3-di-O-methyl-β-D-glucopyranosyluronic acid)-(1→4)-(2,3,6-tri-O-sulfonato-α-D-glucopyranosyl)-(1→4)-(2,3-di-O-methyl-α-L-idopyranosyluronic acid)-(1→4)-2,3,6-tri-O-sulfonato-α-D-glucopyranoside.
